(19) United States
(12) Patent Application Publication
(10) Pub. No.: US 2021/0228272 A1
(43) Pub. Date: Jul. 29, 2021

(54) FLUID GEL FOR FIBER OPTIC GAP

(71) Applicant: St. Jude Medical International Holding S.à r.l., Luxembourg (LU)

(72) Inventors: John Hong, Prior Lake, MN (US); Xuan Y. Khieu, Maple Grove, MN (US)

(21) Appl. No.: 17/257,226

(22) PCT Filed: Aug. 6, 2019
(86) PCT No.: PCT/IB2019/056699

§ 371 Date: Dec. 30, 2020

Related U.S. Application Data
(60) Provisional application No. 62/715,514, filed on Aug. 7, 2018.

(51) Int. Cl. A61B 18/14

(52) U.S. Cl. CPC A61B 18/1492; A61B 2018/00577

(57) ABSTRACT

An ablation catheter including a tip coupled to a distal end of a shaft. The tip can include a displacement feature between a proximal portion and a distal portion of the tip. The distal portion can be configured to move with respect to the proximal portion based on the displacement feature. A first optical fiber can be coupled to the proximal portion. A second optical fiber coupled to the distal portion and optically aligned with the first optical fiber. The second optical fiber can be positioned at a distance from the first optical fiber and can be configured to move with respect to the first optical fiber according to a displacement of the distal portion of the tip. A fluid can be located between the first optical fiber and the second optical fiber.

### CROSS-REFERENCE TO RELATED APPLICATIONS

This application claims the benefit of U.S. provisional application No. 62/715,514, filed 7 Aug. 2018, which is hereby incorporated by reference as though fully set forth herein.

### BACKGROUND

### a. Field

This disclosure relates to an elastomeric moisture barrier for a force sensor.

### b. Background Art

For many years, exploration and treatment of various organs or vessels has been possible using catheter-based diagnostic and treatment systems. Such catheters are introduced through a vessel leading to the cavity of the organ to be explored or treated or alternatively can be introduced directly through an incision made in the wall of the organ. In this manner, the patient avoids the trauma and extended recuperation times typically associated with open surgical procedures.

To provide effective diagnosis or therapy, it is frequently necessary to first map the zone to be treated with great precision. One drawback of such previously known mapping systems is that they rely on manual feedback of the catheter and/or impedance measurements to determine when the catheter is properly positioned in the vessel or organ. Those systems do not measure contact forces with the vessel or organ wall or detect contact forces applied by the catheter against the organ or vessel wall that can modify the true wall location. Instead, previously known mapping methods are time-consuming, dependent upon the skill of the clinician, and cannot compensate for artifacts created by excessive contact forces.

Treatment can be employed to the zone to be treated via the same or a different catheter. Depending upon the specific treatment to be applied to the vessel or organ, the catheter may comprise any of a number of end effectors, such as but not limited to RF ablation electrodes, rotary or scissor action cutting heads, laser ablation system, injection or sewing needles, fluid conveyance systems, forceps, manipulators, mapping electrodes, endoscopic vision systems and therapeutic delivery systems such as genetic impregnation devices.

The effectiveness of such end effectors often depends on having the end effector in contact with the tissue of the wall of the organ or vessel. Many previously-known treatment systems include expandable baskets or hooks that stabilize the distal extremity of the catheter in contact with the tissue. Such arrangements, however, can be inherently imprecise due to the motion of the organ or vessel. Moreover, the previously-known systems do not provide the ability to sense the load applied to the distal extremity of the catheter by movement of the tissue wall.

For example, in the case of a cardiac ablation system, at one extreme the creation of a gap between the end effector of the treatment system and the tissue wall can render the treatment ineffective, and inadequately ablate the tissue zone. At the other extreme, if the end effector of the catheter contacts the tissue wall with excessive force, inadvertent puncturing of the tissue resulting in cardiac tamponade can occur.

### BRIEF SUMMARY

The instant disclosure relates to high-density mapping catheter tips and to map-ablate catheter tips for diagnosing and treating cardiac arrhythmias via, for example, RF ablation. In particular, the instant disclosure relates to flexible high-density mapping catheter tips, and to flexible ablation catheter tips that also have onboard high-density mapping electrodes. Some embodiments include irrigation.

In an embodiment, an ablation catheter can include a tip coupled to a distal end of a shaft. The tip can include a displacement feature between a proximal portion and a distal portion of the tip. The distal portion can be configured to move with respect to the proximal portion based on the displacement feature. A first optical fiber can be coupled to the proximal portion. A second optical fiber coupled to the distal portion and optically aligned with the first optical fiber. The second optical fiber can be positioned at a distance from the first optical fiber and can be configured to move with respect to the first optical fiber according to a displacement of the distal portion of the tip.

A fluid can be located between the first optical fiber and the second optical fiber. For instance, the fluid can fill a gap between the first optical fiber and the second optical fiber. In some examples, the fluid can be uniform between the first optical fiber and the second optical fiber. For instance, the fluid can be free of air pockets, free of contaminants, or both. In a example, the fluid can have a refraction index of 1, 1.3, or any value therebetween. In a further example, the fluid can include a viscosity with self-leveling characteristics. In some instances, the fluid can be a gel, such as a silicone gel.

A fluid coupling can enclose a distal end of the first optical fiber and a proximal end of the second optical fiber. The fluid coupling can be configured to contain the fluid between the first optical fiber and the second optical fiber. In some examples, the fluid coupling can be fixedly attached to a distal end of the first optical fiber or a proximal end of the second optical fiber. In an example, the fluid coupling is sealed to at least one of the first optical fiber or the second optical fiber with at least one seal. In a further example, the fluid is adapted to dampen movement between the first optical fiber and the second optical fiber.

In another embodiment, a system for an ablation catheter including a catheter, an optical emitter, an optical sensor, and a microprocessor. The catheter can include a tip coupled to a distal end of a shaft. The tip can include a displacement feature between a proximal portion and a distal portion of the tip. The distal portion can be configured to move with respect to the proximal portion based on the displacement feature. A first optical fiber can be coupled to the proximal portion. A second optical fiber coupled to the distal portion and optically aligned with the first optical fiber. The second optical fiber can be positioned at a distance from the first optical fiber and can be configured to move with respect to the first optical fiber according to a displacement of the distal portion of the tip.

A fluid can be located between the first optical fiber and the second optical fiber. For instance, the fluid can fill a gap between the first optical fiber and the second optical fiber. In some examples, the fluid can be uniform between the first optical fiber and the second optical fiber. For instance, the fluid can be free of air pockets, free of contaminants, or both. In a example, the fluid can have a refraction index of 1, 1.3, or any value therebetween. In a further example, the fluid can include a viscosity with self-leveling characteristics. In some instances, the fluid can be a gel, such as a silicone gel.

A fluid coupling can enclose a distal end of the first optical fiber and a proximal end of the second optical fiber. The fluid coupling can be configured to contain the fluid between the first optical fiber and the second optical fiber. In some examples, the fluid coupling can be fixedly attached to a distal end of the first optical fiber or a proximal end of the second optical fiber. In an example, the fluid coupling is sealed to at least one of the first optical fiber or the second optical fiber with at least one seal. In a further example, the fluid is adapted to dampen movement between the first optical fiber and the second optical fiber.

In an example, the optical emitter can be configured to transmit a transmission signal through the first optical fiber and into the second optical fiber. The optical sensor can be configured to receive a reflection signal based on the reflection of the transmission signal off of the second optical fiber. The microprocessor can be communicatively coupled to the optical sensor. The microprocessor can be configured to calculate a force applied to the distal portion of the tip based on the reflection signal.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a block diagram of a force sensing system, in accordance with embodiments of the present disclosure.

FIG. 2 is an example of an exploded view of a force ablation catheter, in accordance with embodiments of the present disclosure.

FIG. 3 is an example of a side view of a deformable body of a force ablation catheter, in accordance with embodiments of the present disclosure.

FIG. 4 is an example of a cross section view of a deformable body of a force ablation catheter, in accordance with embodiments of the present disclosure.

FIG. 5 is an example of a detailed view of a cross section of a fluid coupling for a force ablation catheter, in accordance with embodiments of the present disclosure.

### DETAILED DESCRIPTION OF EMBODIMENTS

Several embodiments of a force ablation catheter are disclosed herein. In general, the catheter can include a force sensing tip, such as an optical force sensing tip. In particular, the present disclosure relates to catheters including an optical interferometer, such as a Fabry-Pérot interferometer. The optical interferometer of the catheter can be used to detect an amount of force applied to the tip. For instance, the force applied can be correlated to a displacement of the tip. As discussed herein, the interferometer can be used to measure the displacement and accordingly measure the amount of force applied to the tip. In some examples, the catheter can include a deformable body, a first optical fiber, and a second optical fiber. A gap (e.g., Fabry-Pérot cavity) can be located between the first and second optical fiber. Light traveling along the first optical fiber in a first direction can enter the gap. The light can then reflect from an end face of the second fiber and back into the first fiber heading in a second (e.g., generally opposing) direction. In some examples, the light can reflect multiple times between the faces of the first and second optical fibers before traveling back through the first optical fiber (referred to herein as a reflection signal). An optical sensor and a microprocessor can detect and calculate a distance between the first optical fiber and the second optical fiber (e.g., the dimension of the gap between a first end face of the first optical fiber and a second end face of the second optical fiber). For example, the dimension can be calculated based on an interference pattern of the reflection signal, such as the number of constructive or destructive interference waves (also referred to herein as an interference pattern) detected at the optical sensor.

In some instances, contamination, such as moisture or foreign material, within the gap between the optical fibers can degrade or alter the reflection signal received at the optical sensor. The degraded or altered optical signal can result in errors in the force measurement calculated by the interferometer. Error in the measurement of force at the tip can thwart a desired treatment profile for ablation of the tissue. For example, a prescribed amount of ablation may be desired for treatment. The amount of ablation can depend on the force applied to the tissue. Accordingly, an error in the measurement of the applied force at the tip can result in under or over ablating the tissue. In some examples, too much pressure at the tip can result in a puncture of the tissue. In further examples, the gap is inspected for contamination prior to sale or use, such as in the manufacturing process. Accordingly, contamination within the airgap can increase manufacturing scrap, manufacturing cost, warranty returns, or the like.

The subject matter of the present application can overcome some of these challenges. For instance, a fluid (e.g., gel) can fill the gap between the first optical fiber and the second optical fiber to inhibit contamination (e.g., foreign material, particles, or moisture) from entering the gap. In some examples, the fluid can include a viscosity characteristic that is self-leveling. The viscosity characteristic can mitigate the formation of bubbles within the fluid. Accordingly, the composition of the fluid can be uniform between the first optical fiber and the second optical fiber. The fluid can maintain physical or optical characteristics when exposed to changes in humidity levels, temperature, or other environmental factors. For instance, the optical characteristics (e.g., index of refraction) or physical characteristics (e.g., viscosity) of the fluid can fluctuate less than air under environmental changes, such as temperature or humidity. The consistency of the physical or optical characteristics can increase the accuracy of the interferometric measurements, for instance, as compared to an air-filled gap or a gap contaminated with foreign material or moisture. The fluid can have an index of refraction that can be suitable for use with a Fabry-Pérot interferometer, such as an index of refraction similar to air. A fluid coupling can enclose the distal end (first end face) of the first optical fiber and the proximal end (second end face) of the second optical fiber to contain the fluid within the gap. In some examples, the fluid can dampen oscillations between the first optical fiber and the second optical fiber, for instance, oscillations in the dimension between the first end face and the second end face. Reducing the oscillations can increase the accuracy of the force measurements detected using the interferometer. Details of the various embodiments of the present disclosure are described below with specific reference to the figures.

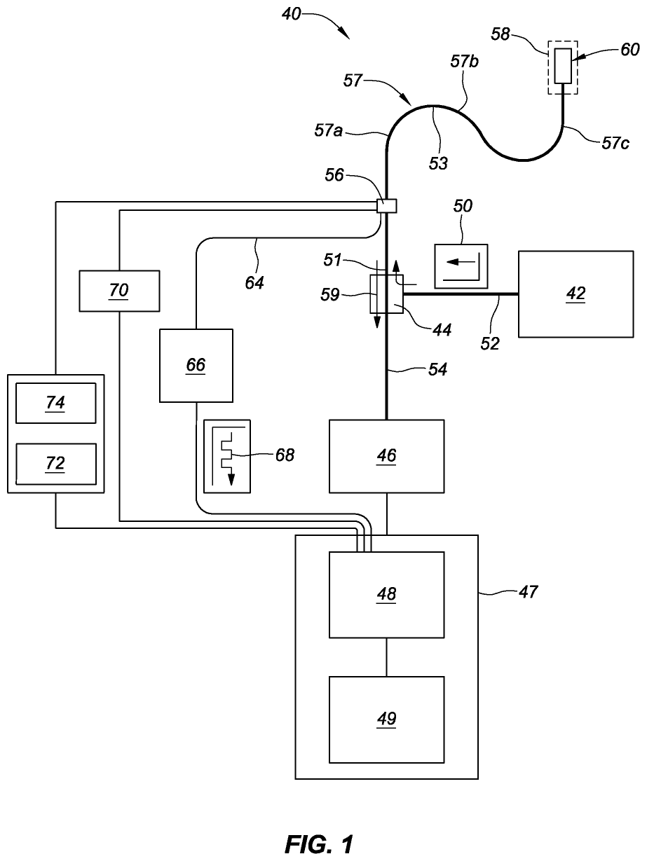

FIG. 1 is a block diagram of a force sensing system 40, in accordance with embodiments of the present disclosure. For example, a force sensing (i.e., contact force) system and force sensor may include technology similar to or the same as that used in the TactiCath™ Quartz™ Ablation Catheter system, commercially available from St. Jude Medical, Inc. of St. Paul, Minn. Additionally, or alternatively, the force sensing system and force sensor may include force sensing sensors, systems, and techniques illustrated and/or described in one or more of U.S. patent application publication nos. 2007/0060847; 2008/0009750; and 2011/0270046, each of which is hereby incorporated by reference in its entirety as though fully set forth herein.

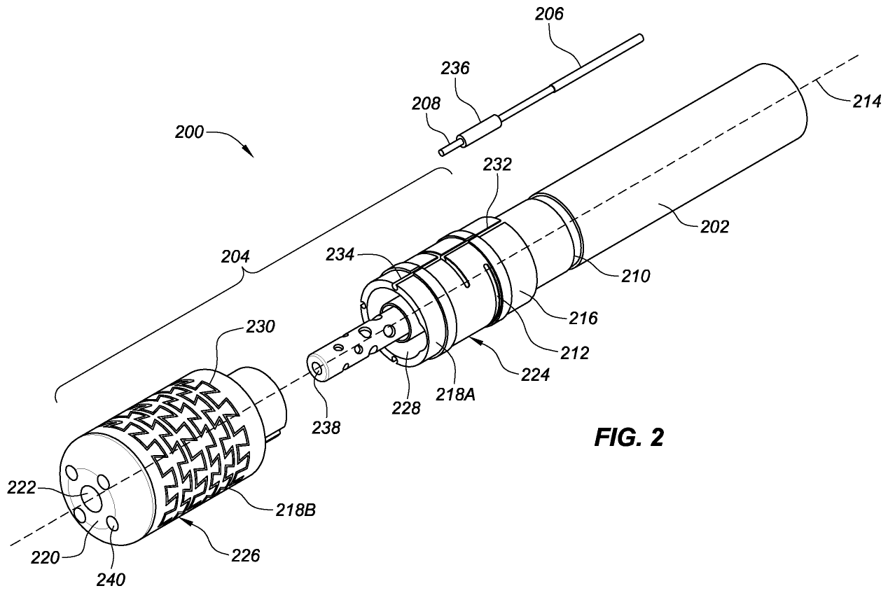

The sensing system 40 can comprise an optical emitter 42 (electromagnetic source), a coupler 44, an optical sensor 46 (receiver), and an operator console 47 including a microprocessor 48 and a computer-readable storage device 49. In some embodiments, the force sensing system 40 can include a plurality of optical fibers for communicating the electromagnetic radiation from the optical emitter 42 to a tip 58 of the catheter assembly 57 and back to an optical sensor 46. As shown in the example of FIG. 1, the plurality of optical fibers can include a first optical fiber 53 (e.g., the first optical fiber as shown in FIG. 2 and described further herein), a transmission fiber 52, and a receiving fiber 54. The optical emitter 42 can be operatively coupled to the proximal end 51 of the first optical fiber 53 through the transmission fiber 52 and the coupler 44. The optical emitter 42 can output a transmission signal 50 of electromagnetic radiation that is substantially steady state in nature, such as a laser or a broadband light source. In some examples, the optical emitter 42 can be a light source, such as a light emitting diode or laser source (e.g., semiconductor laser). The optical sensor 46 can also be operatively coupled to the proximal end 51 (first end face) of the first optical fiber 53 through the coupler 44 and the receiving fiber 54. The optical sensor 46 can include, but is not limited to, a photodiode, charge coupled device (CCD), active pixel sensor (e.g., complementary metal-oxide-semiconductor (CMOS) sensor), or the like. Accordingly, the transmission fiber 52 can transport the electromagnetic radiation to the coupler 44, which directs the transmitted radiation 50 through the first optical fiber 53 (contained within a flexible, elongate catheter assembly 57) to an optical sensing assembly 60 within the tip 58. A reflection signal 59 can be transmitted back through the receiving fiber 54 to the optical sensor 46. In various embodiments of the present disclosure, the optical sensing assembly 60 can detect a contact force exerted on the tip 58. For instance, the tip 58 can include or be configured as an optical interferometer for detecting the displacement of the tip 58 and calculating the corresponding contact force using the microprocessor 48.

The catheter assembly 57 can have a width and a length suitable for insertion into a bodily vessel or organ. In one embodiment, the catheter assembly 57 includes a proximal portion 57a, a middle portion 57b and a distal portion 57c. The distal portion 57c can include a tip 58 that houses the fiber optical sensing assembly 60. The catheter assembly 57 can be of a hollow construction (i.e., having a lumen) or of a non-hollow construction (i.e., no lumen), depending on the application.

In one embodiment, a temperature sensing module 66 including a temperature sensor can be routed through the catheter assembly 57, with a lead line 64 that exits the connector 56. The lead line 64 can be routed to a temperature sensing module 66 that conditions the signal received from the temperature sensor and converts it to a digital signal 68. The digital signal 68 can then be routed to the microprocessor 48 for processing.

The microprocessor 48 can include, but is not limited to, a central processing unit (CPU), graphics processing unit (GPU), microprocessor, application specific integrated circuit (ASIC), a field programmable gate array (FPGA), complementary metal-oxide-semiconductor (CMOS), or the like. In some examples, the computer-readable storage device 49 can include, but is not limited to, random-access memory (RAM), read-only memory (ROM), programmable read-only memory (PROM), erasable programmable read-only memory (EPROM), and electrically erasable programmable read-only memory (EEPROM), dynamic random-access memory (DRAM), static random-access memory (SRAM), Flash memory, or the like.

The microprocessor 48 provides a means for controlling the operation of various components of the sensing system 40, including the catheter assembly 57, an ablation generator 70, and an irrigation system. The microprocessor 48 can also provide a means for determining the position and orientation of the catheter assembly 57 relative to tissue and the body. In some examples, the microprocessor 48 can also provide a means for generating display signals used to control a display.

The catheter assembly 57 can include electrodes configured for ablation. The electrodes can be electrically connected to the ablation generator 70 for delivery of energy, such as radio-frequency (RF) energy, to tissue. In some examples, the catheter assembly 57 can be optionally connected to the irrigation system including a fluid source 72 for delivering a biocompatible irrigation fluid such as saline through a pump 74. The pump 74 can include a fixed rate roller pump or variable volume syringe pump with a gravity feed supply from fluid source 72 as shown. The connector 56 can provide a mechanical, fluid, and electrical connection for conduits or cables extending from the pump 74 and the ablation generator 70. The catheter assembly 57 can also include other conventional components not illustrated herein such as additional electrodes or corresponding conductors or leads.

FIG. 2 is an example of an exploded view of a force ablation catheter 200, in accordance with embodiments of the present disclosure. The catheter 200 can include a shaft 202, a tip 204, and at least one pair of optical fibers (e.g., a first fiber 206 and a second fiber 208). The tip 204 can be located at the distal end 210 of the shaft 202 and can include at least one displacement feature. For instance, the tip 204 can be displaceable with respect to the shaft 202, such as along the longitudinal axis 214 of the shaft 202. In some examples the tip 204 can deflect in directions transverse to the longitudinal axis 214 and along the longitudinal axis 214 of the shaft. The tip 204 can include a proximal portion 216, a distal portion 218 (e.g., distal portion 218A or distal portion 218B), and a plurality of displacement features, such as a displacement feature 212. In the example of FIG. 2, the tip 204 includes two displacement features. The proximal portion 216 can be coupled to the distal end 210 of the shaft 202. The distal portion (e.g., distal portion 218A or distal portion 218B) can include a distal end, such as distal end 220, configured for engaging with tissue. The distal end 220 can include at least one electrode 222 for ablation. The first optical fiber 206 can be fixedly coupled to the proximal portion 216 and the second optical fiber 208 can be fixedly coupled to the distal portion (e.g., distal portion 218A) of the tip 204. Accordingly, the second optical fiber 208 can move with respect to the first optical fiber 206 according to the displacement of the tip 204, such as the displacement of the distal end 220 of the tip 204. A proximal end (first end face) of the first optical fiber can be operatively coupled to an optical sensor, such as the optical sensor 46 shown in FIG. 1 and described herein. A gap between the first optical fiber 206 and the second optical fiber 208 can change dimensions according to the displacement of the tip 204. The optical sensor, first optical fiber 206, second optical fiber 208, and the gap can be configured as an optical interferometer, such as a Fabry-Pérot interferometer, for detecting the amount of force applied at the distal end 220 of the tip 204.

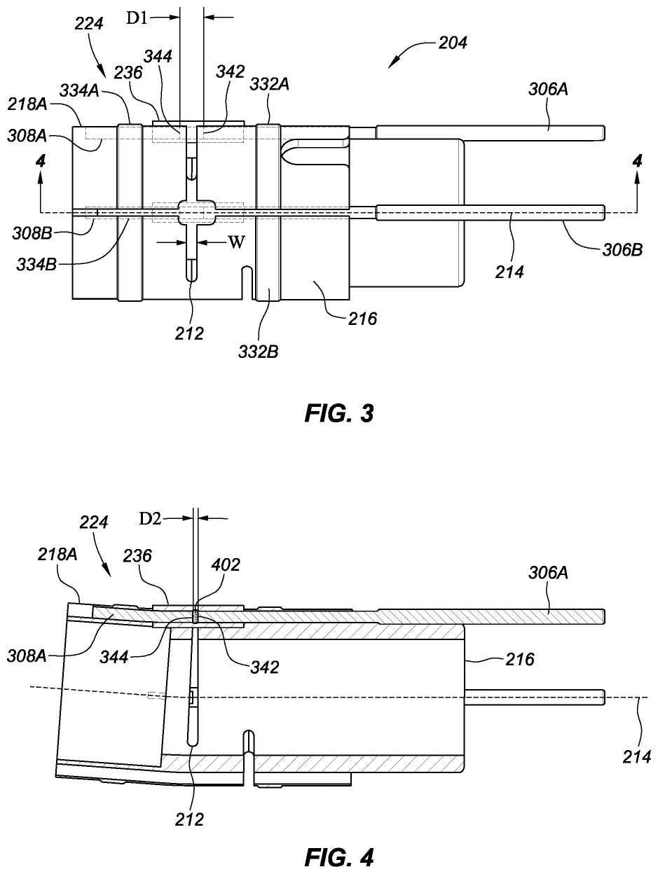

The displacement feature 212 can facilitate movement of the distal portion (e.g., distal portion 218A, distal portion 218B, or both) with respect to the proximal portion 216. In some examples, the displacement feature 212 can be located between the distal portion and the proximal portion 216. For instance, the distal portion can be coupled to the proximal portion by the displacement feature 212. Accordingly, the distal end (e.g., distal end 220) can be displaceable with respect to the proximal portion 216 and the shaft 202. The displacement feature 212 can include, but is not limited to, an elastic element (e.g., rubber biasing element), a flexible element (e.g., a notch flexure as shown in the examples of FIGS. 3 and 4 and described further herein), a telescopic element, a hinge, or the like.

In the example of FIG. 2, the tip 204 can include a deformable body 224 including the displacement feature 212. The proximal portion 216 of the deformable body 224 (e.g., proximal portion of the tip 204) can be coupled to the distal end 210 of the shaft 202. The distal end 218A of the deformable body 224 can be the distal end of the tip 204. In other words, distal end 220 can be included in or integrally formed with the distal portion 218A of the deformable body 224. In a further example, the tip 204 can include an end effector 226 (e.g., distal portion 218B). The end effector 226 can be removably coupled to the deformable body 224 (e.g., the distal portion 218A of the deformable body 224) and can be the distal portion of the tip 204. The deformable body 224 can include an interface 228, such as a socket, for coupling the end effector 226 to the distal end 218A of the deformable body 224. In some instances, the deformable body 224 can be located inside of the end effector 226. The end effector 226 and the deformable body 224 can be compressible based on the displacement of the distal end 220 of the tip 204.

In a further example, the distal portion (e.g., end effector 226) can include one or more articulations 230. The distal end 220 can bend and translate around one or more of the articulations 230 according to force applied at the distal end 220. In the example of FIG. 2, the articulations 230 can be a series of slots within the distal portion, for instance the end effector 226 as shown in FIG. 2. Accordingly, the distal end 220 of the tip can be displaceable based on the articulations 230, the displacement feature 212, or a combination thereof. In various examples, the articulations 230 can include, but are not limited to any hinge, flexure, or the like.

In some examples, the distal portion can be slidable with respect to the proximal portion. For instance, the distal portion can be a shuttle that slides with respect to the proximal portion. In an example, the displacement feature can include or can be combined with a biasing element to urge the distal portion toward an extended or a contracted position.

In an example, the tip 204 can include a first fiber support 232 and a second fiber support 234. The first fiber support 232 can be adapted to hold the first optical fiber 206 and the second fiber support 234 can be adapted to hold the second optical fiber 208. In an example, the first fiber support 232 can be located on the proximal portion 218A and the second fiber support 234 can be located on the distal portion 216. Accordingly, the first optical fiber 206 can be coupled to the proximal portion 216 of the tip 204 and the second optical fiber 208 can be coupled to the distal portion (e.g., distal portion 218A or 218B) of the tip 204. The first fiber and the second fiber can be movable with respect to each other based on the displacement feature 212. The first fiber support 232 or the second fiber support 234 can include, but are not limited to, an interference fit, channel, groove, clamp, grip, sheath, connector, or the like. The first optical fiber 206 and the second optical fiber 208 can be configured to communicate optical signals. In various examples, the first optical fiber 206 or the second optical fiber 208 can be constructed from glass or polymer. In various examples, the first optical fiber 206 or the second optical fiber 208 can be single-mode or multiple-mode fibers.

A fluid coupling 236 can surround (e.g. enclose) the gap between the first optical fiber 206 and the second optical fiber 208. In some examples, the fluid coupling 236 can be attached to one or more of the first optical fiber 206 or the second optical fiber 208. In some examples, the fluid coupling 236 can include, but is not limited to, a metal or plastic sleeve as described further herein. The fluid coupling 236 can be filled with a fluid, such as a gel as described further herein. The fluid can fill the gap between the first optical fiber 206 and the second optical fiber 208. The fluid coupling 236 can be configured to hold the fluid within the gap. In various examples, the fluid coupling 236 can translate along the first optical fiber 206 or the second optical fiber 208 as the gap increases or decreases with movement of the second optical fiber 208 with respect to the first optical fiber 206.

As previously mentioned, the tip 204 can include one or more electrodes 222 for ablating tissue. The one or more electrodes 222 can be located on the distal portion (e.g., 218A or 218B) of the tip 204 or more specifically, in some examples, on the distal end 220 of the tip 204 as shown in the example of FIG. 2. One or more wires can operatively couple the ablation generator (e.g., the ablation generator 70 as shown in FIG. 1) to the ablation electrode 220 for providing energy, such as RF energy, to the ablation electrode 220 for effectuating tissue ablation. The ablation electrode 220 can be a conductive element configured to transmit the energy to the tissue.

In some examples, the catheter can include an irrigation lumen 238. The irrigation lumen 238 can be disposed along the shaft 202, such as along the centerline (e.g., longitudinal axis 214) of the shaft 202 as depicted in the example of FIG. 2. The irrigation lumen 238 can be operatively coupled to the irrigation system (e.g., the irrigation system as shown in the example of FIG. 1 and described herein). In some examples, the tip 204, such as the end effector 226, can include one or more irrigation ports 240 to communicate the irrigation fluid from the irrigation lumen 238 to the tissue. Accordingly, while in operation the irrigation lumen 238 can deliver irrigation fluid to the ablation site to flush and cool the ablated tissue.

FIG. 3 is an example of a side view of the deformable body 224, in accordance with embodiments of the present disclosure. In the example of FIG. 3, the displacement feature 212 can be a notch flexure. For instance, the deformable body 224 includes one or more slots extended partially through the deformable body 224. The displacement feature 212 can reduce the bend strength of the deformable body 224 at a bend location. In an example, a width W of the displacement feature can provide clearance for the deformable body 224 to bend about the displacement feature 212. In the example of FIG. 3, the deformable body 224 is illustrated in an un-displaced configuration. For instance, in the un-displaced configuration, the distal portion 218A of the tip 204 includes a location and shape corresponding to an unloaded state of the deformable body 224.

As previously described, a first optical fiber 306A can be fixedly coupled to the proximal portion 216 of the deformable body 224 and a second optical fiber 308A can be fixedly coupled to the distal portion 218A of the deformable body 224. For instance, the first optical fiber 306A and the second optical fiber 308A (first set of optical fibers) can be located along a first side of the deformable body 224 (e.g., a first lateral side with respect to the longitudinal axis 214 of the deformable body 224). A second set of optical fibers can be located along a different side of the deformable body 224, such as a first optical fiber 306B and a second optical fiber 308B. In some examples, the catheter can include two, three, four, or other number of optical fiber sets. The first optical fiber 306A, 306B can be optically aligned with the second optical fiber 308A, 308B. For instance, a longitudinal axis of the first optical fiber can be aligned with a longitudinal axis of the second optical fiber. Accordingly, an optical signal (e.g., transmitted signal or reflected signal) can be communicated between the first optical fiber and the second optical fiber.

The optical fibers can be fixedly coupled to the deformable body by respective fiber supports. For instance, the first optical fiber 306A and 306B can be coupled to the proximal portion by respective first fiber supports 332A and 332B, and the second optical fiber 308A and 308B can be coupled to the distal portion 218A by respective second fiber supports 334A and 334B. In an example, the fiber supports can be a channel within the deformable body. In some examples, the optical fibers can be attached to the support with an adhesive, an interference fit, or the like.

Accordingly, optical fibers attached to the distal portion 218A of the deformable body 224 (e.g., the second optical fibers 308A, 308B) and the optical fibers attached to the proximal portion 216 of the deformable body (e.g., the first optical fibers 306A, 306B) can be located on opposing sides of the displacement feature 212. The second optical fibers 308A, 308B on the distal portion 218A can move with respect to the first optical fibers 306A, 306B located on the proximal portion 216 when the deformable body 224 is subjected to force resulting in the displacement of the distal end of the deformable body 224, for instance, when the deformable body 224 is bent as shown in the example of FIG. 4. As shown in the example of FIG. 3, the distal end (first end face 342) of the first optical fiber (e.g., 306A) can be located at a distance from the proximal end (second end face 344) of the second optical fiber (e.g., 308A). Accordingly, a gap of dimension D1 can be located between the first end face 342 and the second end face 344.

A coupling, such as the fluid coupling 236, can enclose the gap between the first optical fiber and the second optical fiber for one or more of the sets of optical fibers (e.g., 306A, 306B and 308A, 308B). For instance, the fluid coupling 236 can surround the first end face 342 of the first optical fiber and the second end face 344 of the second optical fiber. In some examples, each of the gaps between the first optical fibers 306A, 308A and the second optical fibers 306B, 308B can be enclosed by a respective fluid coupling, such as fluid coupling 236. In various examples, the fluid coupling 236 can be attached to one or more of the optical fibers. For instance, the fluid coupling 236 can be attached to the first optical fiber 306A and can be slidable along the second optical fiber 308A. In another example, the fluid coupling 236 can be attached to the second optical fiber 308A and can be slidable along the first optical fiber 306A. In some examples, the fluid coupling 236 can enclose more than one gap. For instance, a plurality of first optical fibers (e.g., 306A, B) and a plurality of second optical fibers (e.g., 308A, B), each separated by a respective gap, can have ends that are located within the fluid coupling 236. In various examples, the fluid coupling 236 can be constructed of a material including, but not limited to, metal (e.g., stainless steel), elastomer (e.g., thermoplastic elastomer, polyurethane, polyether block amide, or the like), polymer (e.g., polyamide, polyoxymethylene, polyester, or the like). In some examples, the fluid coupling 236 can be integrated into the deformable body 224. For instance, the deformable body 224 and the fluid coupling 236 can be constructed as a unitary component.

FIG. 4 is an example of a cross section view of a deformable body, such as the deformable body 224 shown in the example of FIG. 3. In the example of FIG. 4, the deformable body 224 is depicted in a displaced configuration. For instance, the distal end 218A of the deformable body 224 can be displaced from the un-displaced configuration when a force is applied to the distal end of the tip (e.g., distal end 220 as shown in the example of FIG. 2). In the displaced configuration, the distal portion 218A can be moved toward the proximal portion 216. In some examples, the distal portion 218A can be translated toward the proximal portion 216, for instance, along the longitudinal axis 214 of the deformable body 224. In other examples, the distal portion 218A can be bent, for instance, about the displacement feature 212. Accordingly, one or more sides (e.g., the upper side, lower side, left side, or right side) can be moved toward the proximal portion 216. In the example of FIG. 4, the deformable body 224 depicts the upper side of the distal portion 218A moved toward the proximal portion 216. The second end face 344 the second optical fiber 308A can be moved toward the first end face 342 of the first optical fiber 306A. Hence, a dimension of the gap D2 between the first end face 342 and the second end face 344 can be decreased with respect to the dimension of the gap D1 in the un-displaced configuration. In further examples, one or more sides (e.g., upper, lower, left, right, or other side) of the deformable body 224 can be moved away from the proximal portion 216. For instance, the distal portion 218A can pivot about the displacement feature 212 to move at least one side of the distal portion 218A toward the proximal portion 216 and at least one side of the distal portion 218A away from the proximal portion 216.

As previously described, the fluid coupling 236 can be filled with a fluid 402, such as a gel. The fluid coupling 236 can contain the fluid 402 between the first optical fiber 306A and the second optical fiber 308A. For instance, the fluid coupling 236 can be sealed against the first optical fiber 306A, the second optical fiber 308A, or both. In an example, the fluid coupling can include one or more seals, such as an O-ring, gasket, adhesive, weld, interference fit, or the like. The fluid 402 can fill the gap between the first optical fiber 306A and the second optical fiber 308A. In some examples, the fluid 402 can be compressible to accommodate a change in the dimension of the gap depending upon the force applied to the tip. In a further example, the fluid coupling 236 can be configured to displace a volume of the fluid corresponding to a decrease in the dimension of the gap. For instance, the fluid coupling 236 can include a cavity sufficiently large to accommodate displaced fluid when the dimension of the gap is decreased. Upon the gap dimension increasing, the fluid 402 can be displaced into the gap so the gap remains filled by the fluid 402.

The first optical fiber (e.g., 306A) and the second optical fiber (e.g., 308A) can be separated by a distance (e.g., distance D1, D2) in one or more of the configurations, or in some examples each configuration, such as in the displaced configuration, the un-displaced configuration, or any configuration therebetween. The change in the dimension of the gap can correspond to the amount of force applied to the distal end (e.g., 220 as shown in the example of FIG. 2) of the tip. In some examples, the fluid 402 can dampen the movement of the second optical fiber with respect to the first optical fiber. For instance, the fluid can reduce oscillations between the relative positions or can decrease the rate of movement between the first optical fiber and the second optical fiber. Reduced oscillations or decreased rate of movement can increase the measurement accuracy of the displacement of the tip and accordingly the measured force.

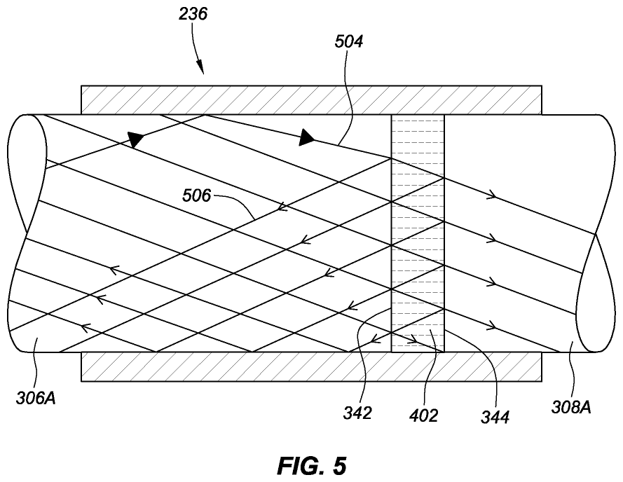

FIG. 5 is an example of a detailed view of a cross section of the fluid coupling, such as the fluid coupling 236, in accordance with embodiments of the present disclosure. As previously described, the fluid 402 can fill the gap between the first optical fiber (e.g., 306A) and the second optical fiber (e.g., 308A) within the fluid coupling 236. The fluid 402 (e.g., gel) can fill the gap between the first optical fiber and the second optical fiber to inhibit contamination (e.g., foreign material, particles, or moisture) from entering the gap. Accordingly, the gap can have reduced contamination based on the presence of the fluid 402. In other words, the gap can be free of contaminants. As described herein, free of contaminants can refer to an amount of contamination that does not interfere with clinically accurate optical measurement of force based on interferometry and is not intended to mean perfectly free of contamination.

The fluid 402 can include, but is not limited to, a gel, such as a silicone gel. The fluid 402 can include optical properties, such as index of refraction between 1 and 1.5, for instance 1 or 1.3. The dimension of the gap can be adjusted to accommodate different indexes of refraction of various fluids. In some examples, the microprocessor (e.g., the microprocessor 48 shown in FIG. 1 and described herein) can be configured to calculate the displacement of the tip and the corresponding force applied to the tip based on a plurality of indexes of refraction for various fluids 402. The fluid 402 can include a viscosity to provide self-leveling characteristics to mitigate bubble or gas pocket formation. For instance, the fluid 402 can include surface tension properties to reduce foaming and bubble formation to provide uniform optical properties between the first optical fiber and the second optical fiber. In an example, the viscosity can reduce leakage from the fluid coupling 236 to keep the fluid 402 within the gap. In various examples, the viscosity of the fluid 402 can include, but is not limited to, a value of 1, 10, 50, 100, 1000, 10000, 100000, 250000 cP, or any value therebetween. In some examples, the viscosity of the fluid can be between 1 cP and 10000 cP, or between 1 cP and 5000 cP. Accordingly, the composition of the fluid 402 can be uniform between the first optical fiber and the second optical fiber. As used herein, a fluid that is sufficiently free of contaminants, bubbles, or other aspects that can interfere with the optical measurements as described herein is considered to be uniform, although the fluid 402 may not be perfectly uniform in practice. In a further example, the fluid 402 can maintain physical or optical characteristics when exposed to changes in humidity levels, temperature, or other environmental factors. For instance, the optical characteristics (e.g., index of refraction) or physical characteristics (e.g., viscosity) of the fluid 402 can fluctuate less than air under environmental changes, such as temperature or humidity. The consistency of the physical or optical characteristics can increase the accuracy of the interferometric measurements, for instance, as compared to an air-filled gap or a gap contaminated with foreign material (e.g., including moisture).

An optical signal (transmission signal 504) can be transmitted from the optical emitter (e.g., the optical emitter 42 shown in FIG. 1) along the first optical fiber (e.g., the first optical fiber 306A) into the gap. An interference pattern can be generated based on the distance of the gap between the first end face 342 and the second end face 344. For instance, the optical signal (transmission signal) can refract off the first end face 342 and reflect off the second end face 344 through the fluid 402 located in the gap. The reflected light can be referred to herein as a reflection signal 506. The transmission signal 504 and the reflection signal 506 can produce constructive and destructive interference resulting in the interference pattern. The interference pattern can be detected by the optical sensor (as shown in the example of FIG. 1 and described herein). The microprocessor (also shown in FIG. 1) can be operatively coupled to the optical sensor and can calculate the distance of the gap based on the interference pattern received at the optical sensor. The change in the dimension of the gap can correspond to the amount of force applied to the distal end of the tip. Accordingly, the displacement of the tip and the corresponding force applied to the tissue can be calculated by the microprocessor. In some examples, the amount of force for effective ablation can be between 100 and 200 grams. Applying the appropriate amount of pressure for ablation can be important to the success of the ablation procedure, for instance, insufficient applied pressure can result in insufficient ablation whereas too much applied pressure can result in excessive ablation or a puncture of the tissue.

Although several embodiments have been described above with a certain degree of particularity, those skilled in the art could make numerous alterations to the disclosed embodiments without departing from the spirit of the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An ablation catheter, comprising:
a shaft including a distal end;
a tip coupled to the distal end of the shaft, the tip including a displacement feature between a proximal portion and a distal portion of the tip, the distal portion configured to move with respect to the proximal portion based on the displacement feature;
a first optical fiber coupled to the proximal portion;
a second optical fiber coupled to the distal portion and optically aligned with the first optical fiber, wherein the second optical fiber is positioned at a distance from the first optical fiber and is configured to move with respect to the first optical fiber according to a displacement of the distal portion of the tip; and
a fluid located between the first optical fiber and the second optical fiber.

2. The ablation catheter of claim 1, wherein the fluid fills a gap between the first optical fiber and the second optical fiber.

3. The ablation catheter of claim 1, further comprising a fluid coupling enclosing a distal end of the first optical fiber and a proximal end of the second optical fiber, the fluid coupling configured to contain the fluid between the first optical fiber and the second optical fiber.

4. The ablation catheter of claim 3, wherein the fluid coupling is fixedly attached to a distal end of the first optical fiber or a proximal end of the second optical fiber.

5. The ablation catheter of claim 3, wherein the fluid coupling is sealed to at least one of the first optical fiber or the second optical fiber with at least one seal.

6. The ablation catheter of claim 1, wherein the fluid is a silicone gel.

7. The ablation catheter of claim 1, wherein the fluid is free of air pockets.

8. The ablation catheter of claim 1, wherein the fluid is free of contaminants.

9. The ablation catheter of claim 1, wherein the fluid is uniform between the first optical fiber and the second optical fiber.

10. The ablation catheter of claim 1, wherein the fluid has a refraction index of between 1 and 1.3.

11. The ablation catheter of claim 1, wherein the fluid includes viscosity with self-leveling characteristics.

12. The ablation catheter of claim 1, wherein the fluid is adapted to dampen movement between the first optical fiber and the second optical fiber.

13. A system for an ablation catheter, the system comprising:
a catheter including:
a shaft including a distal end,
a tip coupled to the distal end of the shaft, the tip including a displacement feature between a proximal portion and a distal portion of the tip, the distal portion configured to move with respect to the proximal portion based on the displacement feature,
a first optical fiber coupled to the proximal portion,
a second optical fiber coupled to the distal portion and optically aligned with the first optical fiber, wherein the second optical fiber is positioned at a distance from the first optical fiber and is configured to move with respect to the first optical fiber according to a displacement of the distal portion of the tip, and
a fluid located between the first optical fiber and the second optical fiber;
an optical emitter configured to transmit a transmission signal through the first optical fiber and into the second optical fiber;
an optical sensor configured to receive a reflection signal based on the reflection of the transmission signal off of the second optical fiber; and
a microprocessor communicatively coupled to the optical sensor, the microprocessor configured to calculate a force applied to the distal portion of the tip based on the reflection signal.

14. The system of claim 13, wherein the fluid fills a gap between the first optical fiber and the second optical fiber.

15. The system of claim 13, further comprising a fluid coupling enclosing a distal end of the first optical fiber and a proximal end of the second optical fiber, the fluid coupling configured to contain the fluid between the first optical fiber and the second optical fiber.

16. The system of claim 15, wherein the fluid coupling is fixedly attached to a distal end of the first optical fiber or a proximal end of the second optical fiber.

17. The system of claim 13, wherein the fluid is a silicone gel.

18. The system of claim 13, wherein the fluid is uniform between the first optical fiber and the second optical fiber.

19. The system of claim 13, wherein the fluid has a refraction index of between 1 and 1.3.

20. The system of claim 13, wherein the fluid is adapted to dampen movement between the first optical fiber and the second optical fiber.
